(19) United States
(12) Patent Application Publication
(10) Pub. No.: US 2022/0249535 A1
(43) Pub. Date: Aug. 11, 2022

(54) COMPOSITIONS COMPRISING NICOTINAMIDE ADENINE DINUCLEOTIDE-RELATED COMPOUNDS AND USE THEREOF

(71) Applicant: Rejuvenation Therapeutics, Incline Village, NV (US)

(72) Inventors: Gary D. ARONSON, Incline Village, NV (US); Sinil KIM, Austin, TX (US)

(21) Appl. No.: 17/601,318

(22) PCT Filed: Apr. 2, 2020
(86) PCT No.: PCT/US2020/026442

§ 371 Date: Oct. 4, 2021

Related U.S. Application Data
(60) Provisional application No. 62/829,975, filed on Apr. 5, 2019.

(51) Int. Cl. A61K 31/7084; A61K 31/706; A61K 31/60; A61K 31/455; A23L 33/10

(52) U.S. Cl. CPC A61K 31/7084; A61K 31/706; A23L 33/10; A61K 31/455; A61K 31/60

(57) ABSTRACT

The present disclosure relates to compositions comprising Nicotinamide Adenine Dinucleotide (NAD+)-related and precursor compounds, pharmaceutical compositions and kits containing them, and methods for using them.

### CROSS-REFERENCE TO RELATED APPLICATIONS

This application claims priority benefit to U.S. Provisional Application No. 62/829,975, filed on Apr. 5, 2019, titled “COMPOSITIONS COMPRISING NICOTINAMIDE ADENINE DINUCLEOTIDE-RELATED COMPOUNDS AND USE THEREOF”, which is incorporated herein by reference for all purposes.

### FIELD OF INVENTION

The present disclosure relates generally to compositions comprising Nicotinamide Adenine Dinucleotide (NAD+)-related and precursor compounds, or a salt thereof, and methods for using them. These methods include, but are not limited to, the enhancement and maintenance of muscle function or growth, treatment of mitochondrial diseases, and the extension of lifespan.

### BACKGROUND

Nicotinamide adenine dinucleotide (NAD+ or NADH) has important biological functions in cellular metabolism. Decline of NAD+ levels has been attributed to the development of many diseases and conditions associated with metabolic dysfunction. Compounds such as nicotinamide, nicotinamide riboside, nicotinic acid, nicotinic acid adenine dinucleotide, nicotinic acid mononucleotide, nicotinamide mononucleotide (NMN), and nicotinic acid riboside (NAR) are involved in NAD+ biosynthesis and metabolism; thus, they can be used to modulate NAD+ levels. Additionally, NAD+ can be further converted to nicotinamide adenine dinucleotide phosphate (NADP+ or NADPH), which also plays a role in cellular metabolism.

Provided herein are compositions comprising NAD+-related and precursor compounds, which may provide benefits for the treatment and prevention of mitochondrial diseases and the extension of lifespan.

### BRIEF SUMMARY

Provided herein is a composition comprising two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing. In some embodiments, the composition further comprises salicylic acid or a salt thereof, or acetylsalicylic acid or a salt thereof. In some embodiments, at least one of the two or more active agents is at least about 60%, about 65%, about 70%, about 75%, about 80%, about 85%, or about 90% pure, wherein the % purity excludes the weight of any other active agents and any added vehicle, diluent, excipient, or carrier. In some embodiments, the composition is sterilized. In some embodiments, the composition further comprises a pharmaceutically acceptable carrier, excipient, binder, or diluent. In some embodiments, the composition further comprises a wax matrix.

Also provided here is a pharmaceutical composition comprising a therapeutically effective amount of a composition, or a pharmaceutically acceptable salt thereof, and a pharmaceutically acceptable carrier, excipient, binder, or diluent, wherein the composition comprises two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing. Also provided here is a food product or dietary supplement comprising a composition or a pharmaceutical composition described herein.

Also provided here is a kit comprising two or more components, wherein each component comprises one or more active agents independently selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing. In some embodiments, the kit further comprises salicylic acid or a salt thereof, or acetylsalicylic acid or a salt thereof. In some embodiments, at least one active agent is at least about 60%, about 65%, about 70%, about 75%, about 80%, about 85%, or about 90% pure, wherein the % purity excludes the weight of any other active agents and any added vehicle, diluent, excipient, or carrier. In some embodiments, at least one of the two or more components is sterilized.

Also provided here is a method of modulating blood NAD+ level in a subject comprising co-administering to the subject a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing. In some embodiments, the method further comprises administering salicylic acid or a salt thereof, or acetylsalicylic acid or a salt thereof. In some embodiments, acetylsalicylic acid or a salt thereof is administered prior to administering other active agents. In some embodiments, at least one of the two or more active agents is at least about 60%, about 65%, about 70%, about 75%, about 80%, about 85%, or about 90% pure, wherein the % purity excludes the weight of any other active agents and any added vehicle, diluent, excipient, or carrier. In some embodiments, at least one of the two or more active agents is sterilized.

In another aspect, the disclosure provides a method of extending lifespan of a subject comprising co-administering to a subject an effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the forgoing. In some embodiments, the method further comprises administering salicylic acid or a salt thereof, or acetylsalicylic acid or a salt thereof. In some embodiments, acetylsalicylic acid or a salt thereof is administered prior to administering other active agents. In some embodiments, at least one of the two or more active agents is at least about 60%, about 65%, about 70%, about 75%, about 80%, about 85%, or about 90% pure, wherein the % purity excludes the weight of any other active agents and any added vehicle, diluent, excipient, or carrier. In some embodiments, at least one of the two or more active agents is sterilized.

In another aspect, the disclosure provides is a method of improving healthspan of a subject comprising co-administering to a subject an effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing. In some embodiments, the method further comprises administering salicylic acid or a salt thereof, or acetylsalicylic acid or a salt thereof. In some embodiments, acetylsalicylic acid or a salt thereof is administered prior to administering other active agents. In some embodiments, at least one of the two or more active agents is at least about 60%, about 65%, about 70%, about 75%, about 80%, about 85%, or about 90% pure, wherein the % purity excludes the weight of any other active agents and any added vehicle, diluent, excipient, or carrier. In some embodiments, at least one of the two or more active agents is sterilized.

In another aspect, the disclosure provides a method of enhancing or maintaining muscle growth or performance comprising co-administering to a subject in need thereof an effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing. In some embodiments, the method further comprises administering salicylic acid or a salt thereof, or acetylsalicylic acid or a salt thereof. In some embodiments, acetylsalicylic acid or a salt thereof is administered prior to administering other active agents. In some embodiments, at least one of the two or more active agents is at least about 60%, about 65%, about 70%, about 75%, about 80%, about 85%, or about 90% pure, wherein the % purity excludes the weight of any other active agents and any added vehicle, diluent, excipient, or carrier. In some embodiments, at least one of two or more active agents is sterilized.

In another aspect, the disclosure provides a method of treating or preventing a mitochondrial disease comprising administering to a subject in need thereof a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing. In some embodiments, the method further comprises administering salicylic acid or a salt thereof, or acetylsalicylic acid or a salt thereof. In some embodiments, acetylsalicylic acid or a salt thereof is administered prior to administering other active agents. In some embodiments, at least one of the two or more active agents is at least about 60%, about 65%, about 70%, about 75%, about 80%, about 85%, or about 90% pure, wherein the % purity excludes the weight of any other active agents and any added vehicle, diluent, excipient, or carrier. In some embodiments, at least one of two or more active agents is sterilized.

### BRIEF DESCRIPTION OF THE DRAWINGS

FIG. 1 depicts the increasing NAD+ level for a subject in response to the intake of (1) 300 mg nicotinamide riboside (NR) and 300 mg nicotinic acid (NA) daily for 11 days followed by 300 mg NR, 300 mg NA, and 1000 mg nicotinamide (NAM) daily for 11 days; (2) 1000 mg NR daily for 7 days; (3) 1000 mg nicotinamide mononucleotide (NMN) daily for 7 days; (4) 1000 mg NAM daily for 14 days; (5) NA 300 mg daily for 14 days; and (6) 1000 mg NAD+ daily for 7 days. Each line represents the NAD+ level of an individual subject tested.

FIG. 2 depicts the increasing NAD+ level in a subject in response to the intake of (1) 300 mg NR and 300 mg NA daily for 11 days followed by 300 mg NR, 300 mg NA, and 1000 mg NAM daily for 11 days; (2) 1000 mg NR daily for 7 days; (3) 300 mg NR daily for 14 days and then 1000 mg NAM daily added for another 7 days; (4) 1000 mg NAM daily for 14 days; and (5) 1000 mg NA and 300 mg NR daily for 11 days. Each line represents the NAD+ level of an individual subject tested.

FIG. 3 compares the increasing NAD+ level in a subject in response to the intake of (1) 1000 mg NR daily for 7 days; (2) 300 mg NR daily and 1000 mg NAM daily for 14 days; (3) NR 300 mg daily for 14 days; (4) 1000 mg NAM daily for 14 days; and (5) 300 mg NA daily and 300 mg NR for 1.3 days (stopped due to flushing). Each line represents the NAD+ level of an individual subject tested.

FIG. 4 depicts increasing NAD+ levels in response to the intake of (A) 1000 mg nicotinamide riboside (NR) daily for 7 days (N=3); (B) 1000 mg nicotinamide mononucleotide (NMN) daily for 7 days (N=3); (C) 1000 mg NAD daily for 7 days (N=4); (D) 1000 mg nicotinamide (NAM) daily for 14 days (N=2); and (E) 300 mg nicotinic acid (NA) daily for 14 days (N=1).

FIG. 5 depicts increasing NAD+ levels in response to the intake of (A) 300 mg NR and 300 NA daily for 11 days followed by 300 mg NR, 300 mg NA, and 1000 mg NAM daily for 11 days (N=1); and (B) 300 mg NR and 1000 NAM daily for 9 days (N=1)

### DETAILED DESCRIPTION

The following description is presented to enable a person of ordinary skill in the art to make and use the various embodiments. Descriptions of specific compositions, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as an antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Except as otherwise noted, the methods and techniques of the present embodiments are generally performed according to conventional methods well known in the art and as described in various general and more specific references that are cited and discussed throughout the present specification.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combinations. All combinations of the embodiments pertaining to particular method steps, reagents, or conditions are specifically embraced by the present disclosure and are disclosed herein just as if each and every combination was individually and explicitly disclosed.

As used herein, a “pharmaceutically acceptable salt” is a salt form that is non-toxic, biologically tolerable, or otherwise biologically suitable for administration to the subject. See generally Berge et al. (1977) J. Pharm. Sci. 66, 1-19. Particular pharmaceutically acceptable salts are those that are pharmacologically effective and suitable for contact with tissues of the subjects without undue toxicity, irritation, or allergic response.

### Composition

In some aspect, provided herein is a composition comprising two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

### NAD+-Related Compounds and Precursors

NAD+ has important functions in cellular bioenergetics and adaptive stress responses. Decreasing NAD+ levels have been associated with metabolism-related diseases, including but not limited to, neurodegenerative diseases, cardiovascular diseases, muscle atrophy, and age-related conditions. Maintenance of NAD+ levels is especially important for cells with higher energy demands. Therefore, molecules involved in the NAD+ biosynthesis and consumption are of particular interest in modulating the NAD+ level as a way to combat these diseases and conditions.

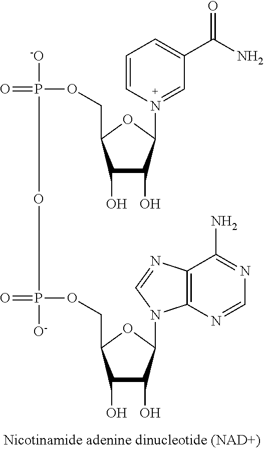

NAD+ can be synthesized from a variety of sources. Major precursors for NAD+ biosynthesis include, but are not limited to, nicotinic acid (NA), nicotinamide riboside (NR), nicotinamide mononucleotide (NMN), nicotinamide (NAM), nicotinic acid adenine dinucleotide, nicotinic acid mononucleotide, and nicotinic acid riboside (NAR). Common food sources for NA include, without limitation, eggs, fish, meat, diary, certain vegetables, and whole grains. NR can be found in milk, and NMN can be found in broccoli, avocado, and beef. Downstream metabolism of ingested NAD+ can also produce these precursors. Depending on the bioavailability of these precursors, NAD+ can be biosynthesized from NA in the Preiss-Handler pathway; or it can be produced from NAM, NR, and NMN in the salvage pathway. In the Preiss-Handler pathway, NA can be converted to nicotinic acid mononucleotide, and then to nicotinic acid adenine dinucleotide, which can produce NAD+ enzymatically. In the salvage pathway, NR and NAM can be converted to NMN which can then produce NAD+ enzymatically. Fang et al. (2017) Trends Mol. Med. 23, 899. NAR can also be generated from NA through NMN formation and is an integral part of NAD metabolism. Kulikova et al. (2015) J. Bio. Chen. 290, 27124. NAD+ can be further converted to NADPH and NADP, another two major reagents and coenzymes in metabolism, especially in a multitude of redox reactions.

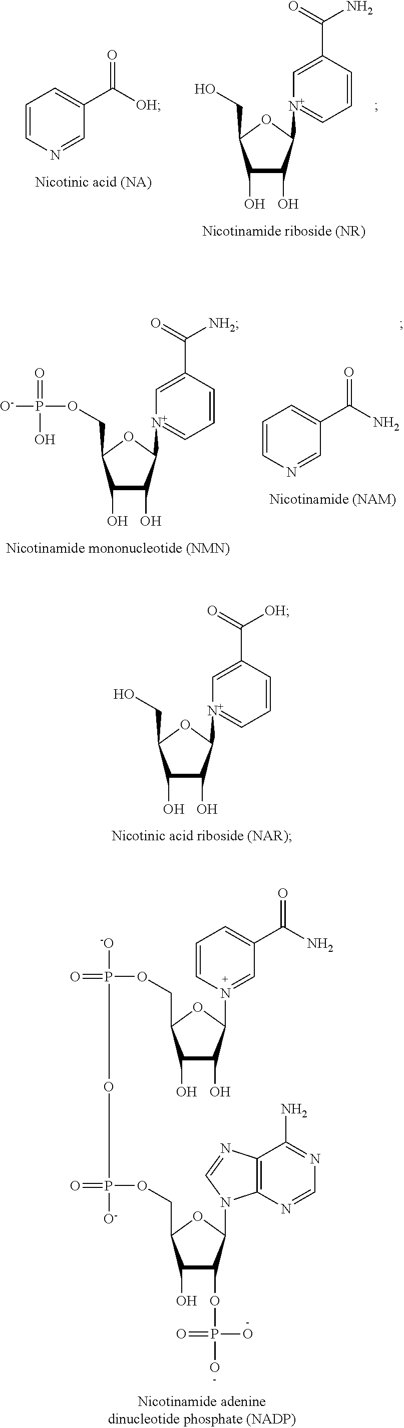

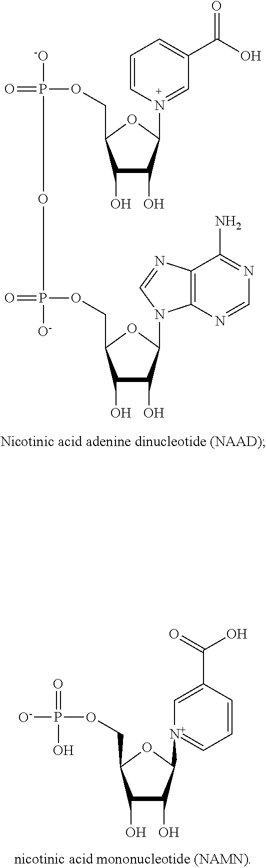

NMN is a direct precursor of NAD+. In common biosynthesis pathways, NR, NAM, and NA need to be converted to NMN in order to produce NAD+. As such, NMN may be a more potent NAD+ modulator compared to the other NAD+ precursors. In some cases, NMN is extracellularly degraded to NR prior to the cellular active transport uptake. See e.g., Nikiforov et al. (2011) J. Biol. Chem. 286, 21767; Grozio et al. (2013) J. Biol. Chem. 288, 25938. In some cases, NMN may act as a sustained-release prodrug formulation for NR, extending its pharmacokinetic life and pharmacodynamics activity over time. See e.g., Grozio et al. (2013) J. Biol. Chem. 288, 25938; Sociali et al. (2016) Oncotarget 7, 2968. After NMN supplementation, NMN can make its way through the liver and bloodstream intact into muscle and is metabolized to NAD+ within 30 minutes. Mills et al. (2016) Cell Metab. 24, 795. And NMN was shown to be retained in the body for longer than NAM. Kawamura et al. (2016) J. Nutr. Sci. Vitaminol 62, 272. Additionally, NMN is abundant in certain vegetables and meat, while chemical synthesis is required to obtain large scales of NR, during which toxic organic solvents are commonly used. See e.g., US2017/0121746. It has been reported that NR, unlike NMN, is unstable under certain conditions and can quickly degrade into NAM in murine plasma or a fetal-bovine-serum containing culture medium. Ratajczak et al. (2016) Nat. Commun. 7, 13103. As such, there may be benefits to the use of NMN, NA, NAR, NAM, nicotinic acid adenine dinucleotide, nicotinamide riboside, nicotinic acid mononucleotide, NAD+/NADH, and/or NADP/NADPH to modulate NAD+ levels.

In another aspect, NAD+-related compounds and precursors are provided as metabolites of other compounds, including but not limited to, inositol hexanicotinate (also known as inositol hexaniacinate or inositol nicotinate). Inositol hexanicotinate is the hexanicotinic acid ester of meso-inositol with the structure shown below. Inositol occurs naturally in the human body and can be made synthetically. When consumed, inositol hexanicotinate can be absorbed and subsequently hydrolyzed in the body to produce nicotinic acid and inositol. Inositol hexanicotinate has been formulated to increase patient tolerability and reduce side effects associated with direct administration of nicotinic acid. Inositol hexanicotinate has been used for treating blood circulation problems such as intermittent claudication, stasis dermatitis, Raynaud's disease, cerebral vascular disease, high blood pressure, and high cholesterol. The extent of hydrolysis of inositol hexanicotinate varies. Without being bound by theory, the composition comprises inositol hexanicotinate that can be intact, partially hydrolyzed, or fully hydrolyzed.

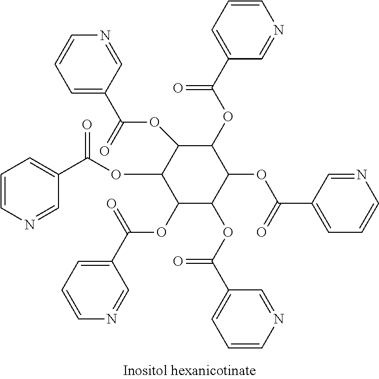 [SMILES: O=C(OC1C(OC(=O)c2cccnc2)C(OC(=O)c2cccnc2)C(OC(=O)c2cccnc2)C(OC(=O)c2cccnc2)C1OC(=O)c1cccnc1)c1cccnc1]

In some embodiments, the composition comprises nicotinamide, nicotinic acid, and nicotinic acid riboside. In some embodiments, the composition comprises NAD+, NADH, NADP+, and NADPH. In some embodiments, the composition comprises nicotinic acid, nicotinamide mononucleotide, and NAD+. In some embodiments, the composition comprises nicotinamide, nicotinamide mononucleotide, and NAD+. In some embodiments, the composition comprises nicotinamide mononucleotide and NAD+. In some embodiments, the composition comprises nicotinic acid riboside and NAD+. In some embodiments, the composition comprises nicotinic acid adenine dinucleotide, nicotinic acid mononucleotide, and nicotinic acid. In some embodiments, the composition comprises nicotinamide riboside and nicotinic acid. In some embodiments, the composition comprises nicotinamide riboside, nicotinamide mononucleotide, and nicotinic acid. In some embodiments, the composition comprises nicotinamide riboside and nicotinamide mononucleotide. In some embodiments, the composition comprises nicotinamide riboside, nicotinic acid, and nicotinamide. In some embodiments, the composition comprises nicotinamide riboside and nicotinic acid riboside. In some embodiments, the composition comprises nicotinamide riboside, NADP+, and NADPH. In some embodiments, the composition comprises nicotinamide riboside, NAD+, and NADH. In some embodiments, the composition comprises nicotinamide riboside, nicotinic acid adenine dinucleotide. In some embodiments, the composition comprises nicotinamide riboside and nicotinic acid mononucleotide. In some embodiments, the composition comprises nicotinamide riboside, nicotinic acid adenine dinucleotide, and nicotinic acid mononucleotide. In some embodiments, the composition comprises NAD+, NADH, NADP, and NADPH. In some embodiments, the composition comprises nicotinic acid, nicotinamide mononucleotide, NADH, and NAD+. In some embodiments, the composition comprises nicotinamide, nicotinamide mononucleotide, NADH, and NAD+. In some embodiments, the composition comprises nicotinamide mononucleotide, NADH, and NAD+. In some embodiments, the composition comprises nicotinic acid riboside, NADH, and NAD+. In some embodiments, the composition comprises NADP and NADPH. In some embodiments, the composition comprises NADH and NAD+. In some embodiments, the composition comprises nicotinic acid adenine dinucleotide, nicotinic acid mononucleotide, and nicotinic acid. In some embodiments, the composition comprises nicotinic acid adenine dinucleotide, nicotinic acid mononucleotide, nicotinamide riboside, NADH, and NAD+.

In some embodiments, the composition comprises nicotinamide, inositol hexanicotinate, and nicotinic acid riboside. In some embodiments, the composition comprises inositol hexanicotinate, nicotinamide mononucleotide, and NAD+. In some embodiments, the composition comprises nicotinic acid adenine dinucleotide, nicotinic acid mononucleotide, and inositol hexanicotinate. In some embodiments, the composition comprises nicotinamide riboside and inositol hexanicotinate. In some embodiments, the composition comprises nicotinamide riboside, nicotinamide mononucleotide, and inositol hexanicotinate. In some embodiments, the composition comprises nicotinamide riboside, inositol hexanicotinate, and nicotinamide. In some embodiments, the composition comprises inositol hexanicotinate, nicotinamide mononucleotide, NADH, and NAD+. In some embodiments, the composition comprises nicotinic acid adenine dinucleotide, nicotinic acid mononucleotide, and inositol hexanicotinate.

In one aspect, the composition comprises a salt of NAD+, NADH, NADP, NADPH, nicotinic acid adenine dinucleotide, nicotinamide riboside, nicotinic acid mononucleotide, nicotinamide, nicotinic acid, nicotinamide mononucleotide, inositol hexanicotinate, or nicotinic acid riboside. In some embodiments, the salt is a pharmaceutically acceptable salt. In some embodiments, pharmaceutically acceptable salts include acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include, without limitation, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include, without limitation, those derived from inorganic bases such as sodium, potassium, lithium, ammonium, calcium, magnesium, iron, zinc, copper, manganese, aluminum salts, and the like. In some embodiments, the organic non-toxic bases are L-amino acids, such as L-lysine and L-arginine, tromethamine, N-ethylglucamine, and N-methylglucamine. Acceptable inorganic bases include, without limitation, aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

In one aspect, the composition comprises salts of one or more of NAD+, NADH, NADP, NADPH, nicotinic acid adenine dinucleotide, nicotinic acid mononucleotide, nicotinamide riboside, inositol hexanicotinate, nicotinamide, nicotinic acid, nicotinamide mononucleotide, or nicotinic acid riboside. In some embodiments, the salt is a pharmaceutically acceptable salt. In some embodiments, the composition comprises salts of one or more of NAD+, NADH, NADP, or NADPH. In some embodiments, the composition comprises salts of one or more of nicotinic acid, nicotinamide mononucleotide, nicotinamide riboside, or NAD+. In some embodiments, the composition comprises salts of one or more of nicotinamide, nicotinamide mononucleotide, nicotinamide riboside, or NAD+. In some embodiments, the composition comprises salts of one or more of nicotinamide mononucleotide or NAD+. In some embodiments, the composition comprises salts of nicotinic acid riboside and NAD+. In some embodiments, the composition comprises salts of nicotinamide riboside, NAD+, NADH, NADP, and NADPH. In some embodiments, the composition comprises salts of nicotinic acid, nicotinamide mononucleotide, NADH, and NAD+. In some embodiments, the composition comprises salts of nicotinamide, nicotinamide mononucleotide, NADH, and NAD+. In some embodiments, the composition comprises salts of nicotinamide mononucleotide, nicotinamide riboside, NADH, and NAD+. In some embodiments, the composition comprises salts of nicotinic acid riboside, nicotinamide riboside, NADH, and NAD+. In some embodiments, the composition comprises salts of nicotinamide, NADH, and NAD+. In some embodiments, the composition comprises salts of nicotinamide mononucleotide, nicotinamide riboside, NADP, and NADPH. In some embodiments, the composition comprises salts of nicotinic acid adenine dinucleotide, nicotinic acid mononucleotide, and nicotinic acid. In some embodiments, the composition comprises salts of nicotinic acid adenine dinucleotide, nicotinic acid mononucleotide, NADH, and NAD+. In some embodiments, the composition comprises salts of nicotinamide riboside and nicotinic acid. In some embodiments, the composition comprises salts of nicotinamide riboside, nicotinamide, and nicotinic acid. In some embodiments, the composition comprises salts of nicotinamide riboside, nicotinamide mononucleotide, and nicotinic acid.

In some embodiments, the composition comprises salts of one or more of inositol hexanicotinate, nicotinamide mononucleotide, nicotinamide riboside or NAD+. In some embodiments, the composition comprises salts of inositol hexanicotinate, nicotinamide mononucleotide, NADH, and NAD+. In some embodiments, the composition comprises salts of nicotinic acid adenine dinucleotide, nicotinic acid mononucleotide, and inositol hexanicotinate. In some embodiments, the composition comprises salts of nicotinamide riboside and inositol hexanicotinate. In some embodiments, the composition comprises salts of nicotinamide riboside, nicotinamide, and inositol hexanicotinate. In some embodiments, the composition comprises salts of nicotinamide riboside, nicotinamide mononucleotide, and inositol hexanicotinate.

In some embodiments, the composition comprises two active agents. In some embodiments, the ratio of the first active agent and the second active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the second active agent is about 1:1 to 25:1 (w/w). In some embodiments, the composition comprises three active agents. In some embodiments, the ratio of the first active agent and the second active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the second active agent is about 1:1 to 25:1 (w/w). In some embodiments, the ratio of the first active agent and the third active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the third active agent is about 1:1 to 25:1 (w/w).

In some embodiments, the composition comprises a combined amount of two and more active agents effective to elevate NAD+ above natural levels within the subject. In some embodiments, the composition comprises a combined amount of two and more active agents effective to treat or prevent a disease or condition that can benefit from elevated NAD+ above natural levels within the subject. In some embodiments, the composition comprises a combined amount of two and more active agents effective to elevate NAD+ above natural levels to increase muscle growth or muscle performance. In some embodiments, the composition comprises a combined amount of two and more active agents effective to elevate NAD+ above natural levels to extend lifespan. In some embodiments, the composition comprises a combined amount of two and more active agents effective to induce an increase of the number of mitochondria present in the subject. In some embodiments, the composition comprises a combined amount of two and more active agents effective to induce a decrease of the reactive oxygen species or a decrease of oxidative stress in the subject. In some embodiments, the two and more active agents exhibit a synergistic effect.

### Additional Components

In another aspect, the composition further comprises a nonsteroidal anti-inflammatory drug, including but not limited to, acetylsalicylic acid (e.g. Aspirin), salicylic acid, salicylate, propionic acid derivatives, acetic acid derivatives, enolic acid derivatives, anthranilic acid derivatives, selective COX-2 inhibitors, or sulfonanilides. In some embodiments, the nonsteroidal anti-inflammatory drug is selected from the group consisting of parecoxib sodium, lornoxicam, iguratimod, pranoprofen, naproxen, bucillamine, celecoxib, flurbiprofen, paeonol, rofecoxib, ketorolac, ibuprofen, diclofenac sodium, tenoxicam, ketoprofen, niflumic acid, zaltoprofen, etodolac, nimesulide, benzidamine hydrochloride, phenylbutazone, aceclofenac, carprofen, droxicam, lobenzarit, oxaprozin, leflunomide, piroxicam, ampiroxicam, acemetacin, tinoridine, indometacin, meloxicam, fenbufen, nabumetone, dolasteron, 3,5-dihydroxy-4-isopropylstilbene, methocarbamol, sulindac, bendazac, tiaprofenic acid, loxoprofen sodium, dulcitol, 1-(2,6-dichlorophenyl)-2-indolinone, flunixin meglumin, megenamic acid, clofenamic acid, butibufen, meclofenamate sodium, flufenamic acid, naproxen sodium, cinchophen, florfenicol, bulleyaconitine A, feclobuzone, flufenisal, ethosalamide, sermetacin, oxyphenyl butazone, crassicauline A, choline magnesium trisalicylate, pirprofen, fenoprofen, neocinchophen, bucolome, ethyl 2-amino-6-benzyl-4,5,6,7-tetrahydrothieno[2,3-c]pyridine-3-carboxylate monohydrochloride, diclofenac diethylamine, clofenamic acid, epirizole, auranofin, cinmetacin, cinaproxen, flutiazin, tolmetin sodium, etofenamate, bufexamac, meclofenamic acid, alminoprofen, tolmetin, flunoxaprofen, fluretofen, fenoprofen calcium, (R)-2-flurbiprofen, loxoprofen, furaprofen, feprazone, diclofenac potassium, diflunisal, and benzydamine.

Salicylic acid occurs in plants in the form of free salicylic acid, carboxylated esters, and phenolic glycosides. It can be found in fruits, vegetables, herbs, spices, nuts, and teas. Common sources include, without limitation, canella, curry powder, dry dill, oregano, rosemary, thyme, mustard, apricots, blackberries, blueberries, cantaloupe, dates, guava, raisins, apples, avocados, cherries, red grapes, mandarin, tangelo, champignon, green pepper, olive, mushrooms, tomato, radish, chicory, alfalfa, broccoli, cucumber, fava beans, spinach, sweet potato, almonds, peanuts, pine nuts, macadamia nuts, and pistachios. In some embodiments, the composition further comprises one or more common sources of salicylic acid.

In another aspect, the composition further comprises a fungus, yeast, or yeast extract. In some embodiments, the yeast or yeast extract comprises species from the Saccharomyces genus (e.g., Saccharomyces cerevisiae). In some embodiments, the yeast or yeast extract comprises species from the Kluyveromyces genus. In some embodiments, the yeast or yeast extract comprises species from the Candida genus. In some embodiments, the yeast or yeast extract comprises species from the Pichia genus. In some embodiments, the yeast or yeast extract comprises the Candida-Uchirisu yeast. In some embodiments, the fungus comprises one or more of the fungal species selected from the group consisting of Rhizopus oryzae, Rhizopus microsporus var. oligosporus, and Rhizopus microspores.

In some embodiments, the composition further comprises one or more additional active or inactive agents including, without limitation, vitamins, minerals, amino acids, carbohydrates, proteins, or lipids. Vitamins may be water-soluble (e.g., vitamin C), or they may be water-insoluble (e.g., vitamin A, vitamin D, vitamin E, or vitamin K). Minerals may be essential minerals (e.g., calcium, chloride, chromium, copper, iodine, iron, magnesium, molybdenum, phosphorus, phosphate, potassium, selenium, sodium, or zinc), or they may be non-essential minerals (e.g., sulfur). Carbohydrates include, without limitation, monosaccharides (e.g., fructose, galactose, glucose, mannose, tagatose, or xylose), disaccharides (e.g., isomaltose, isomaltulose, lactose, maltose, sucrose, trehalose, or trehalulose), and sugar alcohols (e.g., erythritol, glycerol, hydrogenated starch hydrolysates, isomalt, lactitol, maltitol, mannitol, sorbitol, or xylitol). Lipids include, without limitation, saturated fatty acids, monounsaturated fatty acids, and polyunsaturated fatty acids (e.g., omega-3, alpha-linolenic acid (ALA), eicosapentaenoic (EPA), docosahexaenoic acid (DHA), omega-6, arachidonic acid (AA), anandamide (N-arachidonoylethanolamine), linoleic acid, or conjugated linoleic acid (CLA)).

In some embodiments, at least one of the two or more active agents is purified. In some embodiments, an active agent is at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or about 100% pure, wherein the % purity excludes the weight of any other active agents and any added vehicle, diluent, excipient, or carrier.

In some embodiments, the composition is sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

### Pharmaceutical Compositions

The present disclosure also relates to a pharmaceutical composition comprising the composition as described above and a pharmaceutically acceptable carrier, excipient, binder, or diluent.

In some embodiments, the pharmaceutical composition comprises one or more pharmaceutically acceptable excipients. A pharmaceutically-acceptable excipient is a substance that is non-toxic and otherwise biologically suitable for administration to a subject. Such excipients facilitate the administration of the compositions described herein and are compatible with the active ingredients. Examples of pharmaceutically-acceptable excipients include but are not limited to stabilizers, lubricants, surfactants, diluents, anti-oxidants, binders, coloring agents, bulking agents, emulsifiers, or taste-modifying agents. In some embodiments, pharmaceutical compositions according to the embodiments are sterile. Pharmaceutical compositions may be prepared using compounding techniques known or that become available to those skilled in the art. Sterile compositions are also contemplated by the embodiments, including compositions that are in accord with national and local regulations governing such compositions.

The pharmaceutical compositions and compositions described herein may be formulated as solutions, emulsions, suspensions, dispersions, or inclusion complexes such as cyclodextrins in suitable pharmaceutical solvents or carriers, or as pills, tablets, lozenges, bars, suppositories, sachets, dragees, granules, powders, powders for reconstitution, or capsules along with solid carriers according to conventional methods known in the art for the preparation of various dosage forms. Pharmaceutical compositions provided herein may be administered by a suitable route of delivery, such as oral, parenteral, rectal, nasal, or topical route, or by inhalation. In some embodiments, the compositions are formulated for intravenous or oral administration.

For oral administration, the pharmaceutical composition may be provided in a solid form, such as a tablet or capsule, or as a solution, emulsion, or suspension. To prepare the oral composition, the pharmaceutical composition may be formulated to yield a dosage of the composition, e.g., from about 0.01 to about 20 mg/kg daily, from about 20 to about 50 mg/kg daily, or from about 50 to about 200 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents, and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compositions described herein may be formulated for rectal administration as a suppository. For parenteral use, including intravenous, intramuscular, intraperitoneal, intranasal, or subcutaneous routes, the agents provided herein may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 2000 μg/kg/minute of the composition admixed with a pharmaceutical carrier over a period ranging from several minutes to several days.

For nasal, inhaled, or oral administration, the compositions or pharmaceutical compositions described herein may be administered using, for example, a spray formulation also containing a suitable carrier.

In some embodiments, for topical applications, the compositions of the present embodiments are formulated as creams or ointments or similar vehicles suitable for topical administration. For topical administration, the pharmaceutical compositions described herein may be mixed with a pharmaceutical carrier at a concentration of about 0.1% to 1%, 1% to 5%, 5% to 10%, 10% to 20%, 20% to 30%, 0.1% to 0.5%, 0.5% to 1%, 1% to 1.5%, 1.5% to 2%, 2% to 2.5%, 2.5% to 5%, 5% to 7.5%, or 7.5% to 10% of drug to vehicle. Another mode of administering the compositions provided herein may utilize a patch formulation to effect transdermal delivery.

In another aspect, the pharmaceutical composition further comprises a matrix. In some embodiments, the matrix is a hydrophilic matrix, including, without limitation, non-ionic soluble cellulose ether (e.g., hydroxypropylmethylcellulose, hydroxypropylcellulose, and hydroxylethylcellulose), non-ionic homopolymers of ethylene oxide (e.g., poly(ethylene oxide)), water soluble natural gums of polysaccharides (e.g., xanthum gum, alginate, and locust bean gum), water swellable, but insoluble, high molecular weight homopolymers and copolymers of acrylic acid optionally crosslinked with polyalkenyl alcohols, polyvinyl acetate, povidone mixture, cross-linked high amylose starch, and ionic methacrylate copolymers. In some embodiments, the matrix is a hydrophobic matrix, including, without limitation, fatty acids, fatty acid esters, fatty alcohols, waxes of natural and synthetic origins with differing melting points, and hydrophobic polymers. In some embodiments, the hydrophobic matrix comprises stearic acid, lauryl, cetyl or cetostearyl alcohol, carnauba wax, beeswax, candelilla wax, microcrystalline wax, low molecular weight polyethylene, ammoniomethacrylate copolymers, ethyl cellulose, cellulose acetate, cellulose acetate butyrate, cellulose acetate propionate, or latex dispersions of insoluble polymers. In some embodiments, the matrix is a lipid type matrix, biodegradable type matrix, or mineral type matrix. In some embodiments, the matrix is part of the outer layer of a tablet. In some embodiments, the wax matrix comprises a polymer. In some embodiments, the polymer is an acrylic polymer.

In some embodiments, the pharmaceutical composition is formulated for extended release or slow release. In some embodiments, the pharmaceutical composition is formulated with a matrix. In some embodiments, the pharmaceutical composition is formulated with a wax matrix. In some embodiments, the wax matrix is vegetable-based. In some embodiments, the wax matrix comprises an acrylic polymer. In some embodiments, the composition comprises sustained-release nicotinic acid (e.g., NiaSpan™).

### Food Product and Dietary Supplement

The present disclosure also relates to a composition in the form of a food product or a dietary supplement.

A food product comprises a substance that can be used or prepared for use as food. A food product may be in a solid or a liquid (e.g., beverage) form. A food product may contain fruits, plants, vegetables, nuts, seeds, or juice, extracts, jam, concentrate, wheat, or alcohol of any of the foregoing. A food product may also contain milk, yogurt, meat, fish, or processed products of any of the foregoing. A food product may be flowers, leaves, or bark of a plant. A food product may be a product prepared from a natural food. A food product may be a medical food, a functional food, a food additive or a nutritional food. A medical food comprises foods that are specially formulated and intended for dietary management of a disease or condition that has distinctive nutritional needs that cannot be met by normal diet alone. Medical foods can be for oral ingestion or tube feeding. A functional food comprises foods that have a potentially positive effect on health beyond basic nutrition. A food additive comprises any substance added to food and its intended use results or may reasonably be expected to result, directly or indirectly, in its becoming a component or otherwise affecting the characteristics of any food. The addition of food additives may be during production, processing, treatment, packaging, transportation or storage of food. A nutritional food comprises foods that provide a high amount of nutrients. In some embodiments, the nutritional foods also comprise few calories.

A dietary supplement comprises a manufactured product intended to supplement the diet. A dietary supplement can be synthetic or natural. A dietary supplement can comprise one component or more than one component in combination. In some embodiments, the dietary supplement is an addition to the human or animal diet, which is not a natural or conventional food. A dietary supplement may comprise vitamins, amino acids, probiotics, minerals, fiber, fatty acids, pigments, polyphenols, lipids, or proteins. In some embodiments, the dietary supplement may be formulated as a pharmaceutical composition as discussed herein. In some embodiments, the dietary supplement is intended to be taken by mouth as a pill, a capsule, a tablet, or liquid. In some embodiments, the dietary supplement comprises a label as being a dietary supplement. In some embodiments, the dietary supplement comprises non-dietary ingredients such as fillers, artificial colors, sweeteners, flavors, or binders.

In some embodiments, the food product or dietary supplement comprises an effective amount of the composition for the treatment or prevention of a condition that can benefit from increased NAD+ levels. In some embodiments, the condition is muscle performance deficiency, muscle growth deficiency, or mitochondrial disease. In some embodiments, the condition is aging. In some embodiments, the food product or dietary supplement comprises a combined amount of the two or more active agents effective to elevate NAD+ above natural levels. In some embodiments, the food product or dietary supplement comprises a combined amount of the two or more active agents effective to treat or prevent a disease or condition that can benefit from elevated NAD+ above natural levels. In some embodiments, the food product or dietary supplement comprises a combined amount of the two or more active agents effective to elevate NAD+ above natural levels to increase muscle growth or muscle performance. In some embodiments, the food product or dietary supplement comprises a combined amount of the two or more active agents effective to elevate NAD+ above natural levels to extend lifespan. In some embodiments, the food product or dietary supplement comprises a combined amount of the two or more active agents effective to induce an increase of the number of mitochondria present in the subject. In some embodiments, the food product or dietary supplement comprises a combined amount of the two or more active agents effective to induce a decrease of the reactive oxygen species or a decrease of oxidative stress in the subject. In some embodiments, the two or more active agents exhibit a synergistic effect.

### Kits

Provided herein are kits comprising two or more components, wherein each component comprises one or more active agents independently selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

In some embodiments, at least one active agent is purified. In some embodiments, an active agent is at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or about 100% pure, wherein the % purity excludes the weight of any other active agents and any added vehicle, diluent, excipient, or carrier.

In some embodiments, at least one active agent is sterile. Sterile components are compounded or manufactured according to pharmaceutical-grade sterilization standards (United States Pharmacopeia Chapters 797, 1072, and 1211; California Business & Professions Code 4127.7: 16 California Code of Regulations 1751, 21 Code of Federal Regulations 211) known to those of skill in the art.

In one aspect, the kits may contain instructions for use in the treatment or prevention of a condition in a subject in need thereof. In some embodiments, the condition can benefit from increased NAD+ levels. In some embodiments, the condition is muscle performance deficiency, muscle growth deficiency, or a mitochondrial disease. In some embodiments, the condition is aging. A kit may further contain any materials or equipment that may be used in the administration of the active agents or components, such as vials, syringes, or IV bags. A kit may also contain sterile packaging.

In another aspect, the kit may contain any compositions described herein. In another aspect, the kit may contain any pharmaceutical compositions described herein. In some embodiments, the kit contains one or more active agents formulated in any manner described herein. In some embodiments, the kit contains two or more active agents formulated in any manner described herein.

In some embodiments, the kit comprises nicotinamide, nicotinic acid, and nicotinic acid riboside. In some embodiments, the kit comprises NAD+, NADH, NADP+, and NADPH. In some embodiments, the kit comprises nicotinic acid, nicotinamide mononucleotide, and NAD+. In some embodiments, the kit comprises nicotinamide, nicotinamide mononucleotide, and NAD+. In some embodiments, the kit comprises nicotinamide mononucleotide and NAD+. In some embodiments, the kit comprises nicotinic acid riboside and NAD+. In some embodiments, the kit comprises nicotinic acid adenine dinucleotide, nicotinic acid mononucleotide, and nicotinic acid. In some embodiments, the kit comprises nicotinamide riboside and nicotinic acid. In some embodiments, the kit comprises nicotinamide riboside, nicotinamide mononucleotide, and nicotinic acid. In some embodiments, the kit comprises nicotinamide riboside, and nicotinamide mononucleotide. In some embodiments, the kit comprises nicotinamide riboside, nicotinic acid, and nicotinamide. In some embodiments, the kit comprises nicotinamide riboside and nicotinic acid riboside. In some embodiments, the kit comprises nicotinamide riboside, NADP+, and NADPH. In some embodiments, the kit comprises nicotinamide riboside, NAD+, and NADH. In some embodiments, the kit comprises nicotinamide riboside and nicotinic acid adenine dinucleotide. In some embodiments, the kit comprises nicotinamide riboside and nicotinic acid mononucleotide. In some embodiments, the kit comprises nicotinamide riboside, nicotinic acid adenine dinucleotide, and nicotinic acid mononucleotide. In some embodiments, the kit comprises NAD+, NADH, NADP, and NADPH. In some embodiments, the kit comprises nicotinic acid, nicotinamide mononucleotide, NADH, and NAD+. In some embodiments, the kit comprises nicotinamide, nicotinamide mononucleotide, NADH, and NAD+. In some embodiments, the kit comprises nicotinamide mononucleotide, NADH, and NAD+. In some embodiments, the kit comprises nicotinic acid riboside, NADH, and NAD+. In some embodiments, the kit comprises NADP and NADPH. In some embodiments, the kit comprises NADH and NAD+. In some embodiments, the kit comprises nicotinic acid adenine dinucleotide, nicotinic acid mononucleotide, and nicotinic acid. In some embodiments, the kit comprises nicotinic acid adenine dinucleotide, nicotinic acid mononucleotide, nicotinamide riboside, NADH, and NAD+. In some embodiments, the kit compromises nicotinamide, nicotinic acid, and nicotinic acid riboside. In some embodiments, the kit comprises NAD+, NADH, NADP, and NADPH. In some embodiments, the kit comprises nicotinic acid, nicotinamide mononucleotide, and NAD+. In some embodiments, the kit comprises nicotinamide, nicotinamide mononucleotide, and NAD+. In some embodiments, the kit comprises nicotinamide mononucleotide and NAD+. In some embodiments, the kit comprises nicotinic acid riboside and NAD+. In some embodiments, the kit comprises nicotinic acid adenine dinucleotide and nicotinic acid mononucleotide.

In some embodiments, the kit comprises nicotinamide, inositol hexanicotinate, and nicotinic acid riboside. In some embodiments, the kit comprises inositol hexanicotinate, nicotinamide mononucleotide, and NAD+. In some embodiments, the kit comprises nicotinic acid adenine dinucleotide, nicotinic acid mononucleotide, and inositol hexanicotinate. In some embodiments, the kit comprises nicotinamide riboside and inositol hexanicotinate. In some embodiments, the kit comprises nicotinamide riboside, nicotinamide mononucleotide, and inositol hexanicotinate. In some embodiments, the kit comprises nicotinamide riboside, inositol hexanicotinate, and nicotinamide. In some embodiments, the kit comprises inositol hexanicotinate, nicotinamide mononucleotide, NADH, and NAD+. In some embodiments, the kit comprises nicotinic acid adenine dinucleotide, nicotinic acid mononucleotide, and inositol hexanicotinate. In some embodiments, the kit compromises nicotinamide, inositol hexanicotinate, and nicotinic acid riboside.

In one aspect, the kit comprises salts of one or more of NAD+, NADH, NADP, NADPH, nicotinic acid adenine dinucleotide, nicotinic acid mononucleotide, nicotinamide riboside, nicotinamide, nicotinic acid, nicotinamide mononucleotide, inositol hexanicotinate, or nicotinic acid riboside. In some embodiments, the salt is a pharmaceutically acceptable salt. In some embodiments, the kit comprises salts of one or more of NAD+, NADH, NADP, or NADPH. In some embodiments, the kit comprises salts of one or more of nicotinic acid, nicotinamide mononucleotide, nicotinamide riboside, or NAD+. In some embodiments, the kit comprises salts of one or more of nicotinamide, nicotinamide mononucleotide, nicotinamide riboside, or NAD+. In some embodiments, the kit comprises salts of at least one of nicotinamide mononucleotide or NAD+. In some embodiments, the kit comprises salts of nicotinic acid riboside and NAD+. In some embodiments, the kit comprises salts of nicotinamide riboside, NAD+, NADH, NADP, and NADPH. In some embodiments, the kit comprises salts of nicotinic acid, nicotinamide mononucleotide, NADH, and NAD+. In some embodiments, the kit comprises salts of nicotinamide, nicotinamide mononucleotide, NADH, and NAD+. In some embodiments, the kit comprises salts of nicotinamide mononucleotide, nicotinamide riboside, NADH, and NAD+. In some embodiments, the kit comprises salts of nicotinic acid riboside, nicotinamide riboside, NADH, and NAD+. In some embodiments, the kit comprises salts of nicotinamide, NADH, and NAD+. In some embodiments, the kit comprises salts of nicotinamide mononucleotide, nicotinamide riboside, NADP, and NADPH. In some embodiments, the kit comprises salts of nicotinic acid adenine dinucleotide, nicotinic acid mononucleotide, and nicotinic acid. In some embodiments, the kit comprises salts of nicotinic acid adenine dinucleotide, nicotinic acid mononucleotide, NADH, and NAD+. In some embodiments, the kit comprises salts of nicotinamide riboside and nicotinic acid. In some embodiments, the kit comprises salts of nicotinamide riboside, nicotinamide, and nicotinic acid. In some embodiments, the kit comprises salts of nicotinamide riboside, nicotinamide mononucleotide, and nicotinic acid.

In some embodiments, the kit comprises salts of one or more of inositol hexanicotinate, nicotinamide mononucleotide, nicotinamide riboside, or NAD+. In some embodiments, the kit comprises salts of inositol hexanicotinate, nicotinamide mononucleotide, NADH, and NAD+. In some embodiments, the kit comprises salts of nicotinic acid adenine dinucleotide, nicotinic acid mononucleotide, and inositol hexanicotinate. In some embodiments, the kit comprises salts of nicotinamide riboside and inositol hexanicotinate. In some embodiments, the kit comprises salts of nicotinamide riboside, nicotinamide, and inositol hexanicotinate. In some embodiments, the kit comprises salts of nicotinamide riboside, nicotinamide mononucleotide, and inositol hexanicotinate.

In some embodiments, the kit comprises a dosage form (e.g., a unit dosage form) having the composition at greater than about 20%, or greater than about 25%, or greater than about 30%, or greater than about 35%, or greater than about 40%, or greater than about 45%, or greater than about 50%, or greater than about 55%, or greater than about 60%, or greater than about 65%, or greater than about 70%, or greater than about 75%, or greater than about 80%, or greater than about 85%, or greater than about 90%, or greater than 95% by weight. In some embodiments, the kit comprises a dosage form (e.g., a unit dosage form) having the composition at less than 20%, or less than about 25%, or less than about 30%, or less than about 35%, or less than about 40%, or less than about 45%, or less than about 50%, or less than about 55%, or less than about 60%, or less than about 65%, or less than about 70%, or less than about 75%, or less than about 80%, or less than about 85%, or less than about 90%, or less than 95% by weight.

In some embodiments, the kit comprises two active agents. In some embodiments, the ratio of the first active agent and the second active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the second active agent is about 1:1 to 25:1 (w/w). In some embodiments, the kit comprises three active agents. In some embodiments, the ratio of the first active agent and the second active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the second active agent is about 1:1 to 25:1 (w/w). In some embodiments, the ratio of the first active agent and the third active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the third active agent is about 1:1 to 25:1 (w/w).

In some embodiments, the kit comprises a combined amount of the two or more components effective to elevate NAD+ above natural levels. In some embodiments, the kit comprises a combined amount of the two or more components effective to treat or prevent a disease or condition that can benefit from elevated NAD+ above natural levels. In some embodiments, the kit comprises a combined amount of the two or more components effective to elevate NAD+ above natural levels to increase muscle growth or muscle performance. In some embodiments, the kit comprises a combined amount of the two or more components effective to elevate NAD+ above natural levels to extend lifespan. In some embodiments, the kit comprises a combined amount of the two or more components effective to induce an increase of the number of mitochondria present in the subject. In some embodiments, the kit comprises a combined amount of the two or more components effective to induce a decrease of the reactive oxygen species or a decrease of oxidative stress in the subject.

In another aspect, the kit further comprises a fungus, yeast or yeast extract. In some embodiments, the yeast or yeast extract comprises species from the Saccharomyces genus. In some embodiments, the yeast or yeast extract comprises species from the Kluyveromyces genus. In some embodiments, the yeast or yeast extract comprises species from the Candida genus. In some embodiments, the yeast or yeast extract comprises species from the Pichia genus. In some embodiments, the yeast or yeast extract comprises the Candida-Uchirisu yeast. In some embodiments, the yeast or yeast extract comprises one or more of the fungal species Rhizopus oryzae, Rhizopus microsporus var. oligosporus, or Rhizopus microsporus.

In some embodiments, the kit further comprises one or more additional active or inactive agents including, without limitation, vitamins, minerals, amino acids, carbohydrates, proteins, or lipids (e.g., as described herein).

In another aspect, the kit comprises a pharmaceutical composition comprising one or more active agents and a pharmaceutically acceptable carrier, excipient, binder or diluent. In another aspect, the kit comprises a pharmaceutical composition comprising two or more active agents and a pharmaceutically acceptable carrier, excipient, binder or diluent.

The active agents described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. The active agents provided herein may be administered by a suitable route of delivery, such as oral, parenteral, rectal, nasal, topical, or ocular routes, or by inhalation. In some embodiments, the active agents are formulated for intravenous or oral administration.

The two or more active agents in the pharmaceutical composition can be formulated separately in two formulations or together in one formulation. In some embodiments, the two or more active agents are formulated together as a solution, emulsion, suspension, dispersion, or inclusion complex such as with cyclodextrins in suitable pharmaceutical solvents or carriers, or as pills, tablets, bars, lozenges, suppositories, sachets, dragees, granules, powders, powders for reconstitution, or capsules along with solid carriers according to conventional methods known in the art for preparation of various dosage forms. In some embodiments, the two or more active agents are formulated separately. In some embodiments, the two or more active agents are formulated in different dosage forms. In some embodiments, at least one active agent is formulated as a liquid, and at least one active agent is formulated as a solid. In some embodiments, at least one active agent is formulated as a solution or suspension, and at least one active agent is formulated as a pill or tablet. The two or more active agents of the kit can be formulated, together or separately, in any manner and with any excipients or other ingredients described herein. In some embodiments, the two or more active agents are both formulated as solids. In some embodiments, the two or more active agents are both formulated as liquids.

In some embodiments, the two or more active agents are formulated for simultaneous administration. In some embodiments, the two or more active agents are formulated for sequential administration. In some embodiments, the two or more active agents are formulated for administration via different routes. In some embodiments, the two or more active agents are administered in the same route (e.g., oral administration). In some embodiments, the administration of at least one active agent and the administration of the other active agents are about 1-60 mins, about 60-120 mins, about 120-240 mins, about 240-480 mins, about 480-1440 mins, about 1-2 days, about 2-5 days, or about 5-10 days apart.

In another aspect, at least one active agent may be in the form of a food product or a dietary supplement. In some embodiments, at least one active agent is administered in the form of a natural food, including fruits, vegetables, plants, meat, milk, nuts, or seeds. In some embodiments, at least one active agent is administered in the form of a processed food product, including juice, extract, concentrate, jam, or alcohol.

In some embodiments, at least one active agent may be formulated for extended release or slow release. In some embodiments, the active agent is formulated with a matrix. In some embodiments, the active agent is formulated with a wax matrix. In some embodiments, the wax matrix is vegetable-based. In some embodiments, the wax matrix comprises an acrylic polymer.

In other aspects, the kits may be used for any of the methods described herein, including, for example, to treat or prevent a mitochondrial disease, to extend lifespan, or to improve healthspan.

In another aspect, kits for treating a subject who suffers from or is susceptible to the conditions described herein are provided, comprising a first container comprising a dosage amount of the two or more active agents as disclosed herein, and instructions for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the subject.

Kits may optionally include appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

Kits may also be provided that contain sufficient dosages of the two or more active agents described herein (including pharmaceutical compositions thereof) to provide effective treatment or prevention for a subject for an extended period, such as about 1-3 days, about 1-5 days, about a week, about 2 weeks, about 3 weeks, about 4 weeks, about 6 weeks, about 8 weeks, about 3 months, about 4 months, about 5 months, about 6 months, about 7 months, about 8 months, about 9 months or more.

Kits may also include multiple doses of the two or more active agents and instructions for use and may be packaged in quantities sufficient for storage and use in pharmacies, for example, hospital pharmacies and compounding pharmacies. In certain embodiments the kits may include a dosage amount of the two or more active agents as disclosed herein.

Kits may include the composition as described herein packaged in either a unit dosage form or in a multi-use form. Kits may also include multiple units of the unit dose form.

Kits may also comprise a means for the delivery of the composition thereof.

### Treatment

The present disclosure also relates to a method for treatment or prevention of a disease or condition that can benefit from increased NAD+ levels, anticancer or anti-inflammatory treatment, or for extending lifespan. The method comprises administering to a subject in need thereof a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

As used herein, the term “treat” or “treatment” refers to an approach for obtaining a beneficial or desired result, including clinical results. For purposes of this disclosure, beneficial or desired results include, but are not limited to: reducing the severity of or suppressing the worsening of an existing disease, symptom, or condition, alleviating a symptom and/or diminishing the extent of a symptom and/or preventing a worsening of a symptom associated with a condition, arresting the development of a disease, symptom, or condition, relieving the disease, symptom, or condition, causing regression of the disease, disorder, or symptom (in terms of severity or frequency of negative symptoms), or stopping the symptoms of the disease or condition. Beneficial or desired results can also be slowing, halting, or reversing the progressive course of a disease or condition.

In one aspect, the two or more active agents are formulated and administered as one composition. In some embodiments, the composition further comprises a pharmaceutically acceptable salt. In some embodiments, at least one active agent is in the form of a food product or a dietary supplement. In some embodiments, the composition further comprises a pharmaceutically acceptable carrier, excipient, binder, or diluent. The two or more active agents provided herein may be administered by a suitable route of delivery, such as oral, parenteral, rectal, nasal, topical, or ocular routes, or by inhalation. In some embodiments, the two or more active agents are formulated for intravenous or oral administration. For oral administration, the two or more active agents may be provided in a solid form, such as a tablet or capsule, or as a solution, emulsion, or suspension.

In another aspect, the two or more active agents are formulated individually and administered simultaneously. In some embodiments, the method comprises a pharmaceutical composition comprising at least one active agent and a pharmaceutically acceptable carrier, excipient, binder, or diluent. In some embodiments, the method comprises a pharmaceutical composition comprising at least two active agents and a pharmaceutically acceptable carrier, excipient, binder, or diluent.

In some embodiments, at least one active agent is purified. In some embodiments, an active agent is at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or about 100% pure, wherein the % purity excludes the weight of any other active agents and any added vehicle, diluent, excipient, or carrier.

In some embodiments, the formulations and preparations used in the methods of the disclosure are sterile. Sterile pharmaceutical formulations are compounded or manufactured according to pharmaceutical-grade sterilization standards (United States Pharmacopeia Chapters 797, 1072, and 1211; California Business & Professions Code 4127.7: 16 California Code of Regulations 1751, 21 Code of Federal Regulations 211) known to those of skill in the art. In some embodiments, at least one active agent is sterilized.

In another aspect, the two or more active agents are formulated individually and administered sequentially. In some embodiments, the two or more active agents are administered via different routes. In some embodiments, the two or more active agents are administered via the same route. In some embodiments, the administration of at least one active agent and the administration of the other active agents are about 1-60 mins, about 60-120 mins, about 120-240 mins, about 240-480 mins, about 480-1440 mins, about 1-2 days, about 2-5 days, or about 5-10 days apart. In some embodiments, at least one active agent is formulated as a pill, tablet, or capsule, while at least one active agent is formulated as a solution or suspension. In some embodiments, at least one active agent is formulated as a food product, while at least one active agent is formulated as a food additive. In some embodiments, at least one active agent is formulated as a dietary supplement, while at least one active agent is formulated as a food product.

In another aspect, the two or more active agents can be formulated as any pharmaceutical compositions described herein.

In some embodiments, the two or more active agents are provided as a kit.

In another aspect, the dosage regimen of the two or more active agents is about 1-10 mg/day, about 10-20 mg/day, about 20-30 mg/day, about 30-40 mg/day, about 40-50 mg/day, about 50-60 mg/day, about 60-70 mg/day, about 70-80 mg/day, about 80-90 mg/day, about 90-100 mg/day, about 100-150 mg/day, about 150-200 mg/day, about 200-250 mg/day, about 250-300 mg/day, about 300-350 mg/day, about 350-400 mg/day, about 400-450 mg/day, about 450-500 mg/day, about 500-600 mg/day, about 600-700 mg/day, about 700-800 mg/day, about 800-900 mg/day, about 900-1000 mg/day, about 1000-1200 mg/day, about 1200-1400 mg/day, about 1400-1600 mg/day, about 1600-1800 mg/day, about 1800-2000 mg/day, about 2000-2200 mg/day, about 2200-2400 mg/day, about 2400-2600 mg/day, about 2600-2800 mg/day, about 2800-3000 mg/day, about 3000-3200 mg/day, about 3200-3400 mg/day, about 3400-3600 mg/day, about 3600-3800 mg/day, about 3800-4000 mg/day, about 4000-4200 mg/day, about 4200-4400 mg/day, about 4400-4600 mg/day, about 4600-4800 mg/day, about 4800-5000 mg/day, or about 5000-10000 mg/day for about 1-7 days, about 1-14 days, about 1-60 days 1-120 days, about 21-240 days, or about 120-364 days.

In some embodiments, the method comprises administering two active agents. In some embodiments, the ratio of the first active agent and the second active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the second active agent is about 1:1 to 25:1 (w/w). In some embodiments, the method comprises administering three active agents. In some embodiments, the ratio of the first active agent and the second active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the second active agent is about 1:1 to 25:1 (w/w). In some embodiments, the ratio of the first active agent and the third active agent is about 1:1 to 5:1, about 5:1 to 10:1, about 10:1 to 20:1, about 20:1 to 30:1, about 30:1 to 40:1, about 40:1 to 50:1, or about 50:1 to 100:1 (w/w). In some embodiments, the ratio of the first active agent and the third active agent is about 1:1 to 25:1 (w/w).

In some embodiments, the dosage of the combined two or more active agents is effective to elevate NAD+ above natural levels. In some embodiments, the dosage of the combined two or more active agents is effective to treat or prevent a disease or condition that can benefit from elevated NAD+ above natural levels. In some embodiments, the dosage of the combined two or more active agents is effective to elevate NAD+ above natural levels to increase muscle growth or muscle performance. In some embodiments, the dosage of the combined two or more active agents is effective to elevate NAD+ above natural levels to extend lifespan. In some embodiments, the dosage of the combined two or more active agents is effective to induce an increase of the number of mitochondria present in the subject. In some embodiments, the dosage of the combined two or more active agents is effective to induce a decrease of the reactive oxygen species or a decrease of oxidative stress in the subject. In some embodiments, the two or more active agents exhibit a synergistic effect.

In another aspect, an effective amount of the two or more active agents is administered to a subject in need thereof. A subject may be a mammalian patient (such as a human or an animal (e.g., cat, dog, cow, rat, mouse, horse, sheep, pig, goat, buffalo, chicken, duck, goose, or other domesticated mammal)), a nematode, a fungus, a eukaryotic cell, or a bacterium. In some embodiments, the subject is C. elegans or yeast.

In another aspect, an effective amount of the two or more active agents is administered to an experimental subject for research purposes. A subject may be a mammalian patient (such as a human or an animal (e.g., mouse, rat, monkey, ape, worm, fly, fruit fly, fish, Zebrafish, frog, Xenopus, cat, dog, cow, pig, horse, sheep, goat, buffalo, chicken, duck, goose or other domesticated mammal)), a nematode, a fungus, a eukaryotic cell, or a bacterium. In some embodiments, the subject is a cell line, cell strain, animal organ, group of cells, C. elegans, or yeast.

In another aspect, the administration of the two or more active agents exhibits a synergistic effect toward treatment or prevention of a disease or condition that can benefit from increased NAD+ levels, anticancer or anti-inflammatory treatment, or for extending lifespan.

In another aspect, the disease or condition to be treated or prevented using any of the compositions or methods described herein includes deficiency in muscle growth or muscle performance or a mitochondrial disease, including, but not limited to, aging, such as Alzheimer's disease, Parkinson's disease, and amyotrophic lateral sclerosis; neurodegenerative disorders, such as Huntington's disease, AIDS dementia complex, adrenoleukodystrophy, Alexander disease, Alper's disease, Batten disease, Bovine spongiform encephalopathy, ataxia telangiectasia, Canavan disease, corticobasal degeneration, Parkinson's disease, Alzheimer's disease, amyotrophic lateral sclerosis, Lyme disease, and multiple sclerosis; metabolic disorders, such as diabetes, obesity, insulin resistance, and a metabolic syndrome, or any combinations thereof.

In some embodiments, the mitochondrial disease or disorder is a neuromuscular disorder, a disorder of neuronal instability, a neurodegenerative disorder, or a mitochondrial myopathy. In yet further embodiments, the mitochondrial disease or disorder is Friedrich's Ataxia, muscular dystrophy, multiple sclerosis, seizure disorders, migraine, Alzheimer's disease, Parkinson's disease, amyotrophic lateral sclerosis, ischemia, renal tubular acidosis, age-related neurodegeneration and cognitive decline, chemotherapy fatigue, frailty, age-related or chemotherapy-induced menopause or irregularities of menstrual cycling or ovulation, mitochondrial myopathies, mitochondrial damage (e.g., calcium accumulation, excitotoxicity, nitric oxide exposure, drug induced toxic damage or hypoxia), mitochondrial deregulation, Creutzfeldt-Jakob disease, dementia with Lewy bodies, fatal familial insomnia, multiple system atrophy, Huntington's disease, Kennedy's disease, frontotemporal lobar degeneration, Machado-Joseph disease, Krabbe disease, neuroacanthocytosis, Pick's disease, Niemann-Pick disease, progressive supranuclear palsy, primary lateral sclerosis, Sandhoff disease, diffuse myelinoclastic sclerosis, Refsum disease, spinocerebellar ataxia, tabes dorsalis, subacute combined degeneration of spinal cord, Tay-Sachs disease, transmissible spongiform encephalopathy, wobbly hedgehog syndrome, toxic encephalopathy, Barth syndrome, beta-oxidation defects, carnitine-acyl-carnitine deficiency, carnitine deficiency, creatine deficiency syndrome, co-enzyme Q10 deficiency, complex I deficiency, complex II deficiency, complex III deficiency, complex IV deficiency, complex V deficiency, chronic progressive external ophthalmoplegia syndrome, CPT I deficiency, CPT II deficiency, Kearns-Sayre syndrome, lactic acidosis, leukodystrophy, Leigh disease, Luft disease, mitochondrial encephalomyopathy lactic acidosis and strokelike episodes (MELAS), progeria, Cockayne syndrome, myoclonic epilepsy and ragged-red fiber disease (MERRF), mitochondrial recessive ataxia syndrome, mitochondrial cytopathy, mitochondrial DNA depletion, mitochondrial encephalopathy, myoneurogastrointestinal disorder and encephalopathy, neuropathy, ataxia, and retinitis pigmentosa, Pearson syndrome, pyruvate carboxylase deficiency, pyruvate dehydrogenase deficiency, POLG2 mutations, encephalopathy and possibly liver disease or cardiomyopathy, acyl-CoA dehydrogenase deficiency, chronic traumatic encephalopathy (CTE), cancer, cachexia, infections caused by Hepatitis A, B, and/or C Virus, Human Immunodeficiency Virus (HIV), Human Papilloma Virus, human T-cell leukemia-lymphoma viruses (HTLV) or, any combinations thereof or any of their medical sequelae.

In some embodiments, the disease is a disease associated with dementia. In certain embodiments, the disease associated with dementia is selected from the group consisting of Alzheimer's disease, vascular dementia, dementia with Lewy bodies (DLB), Parkinson's disease, frontotemporal dementia, Creutzfeldt-Jakob disease, Normal pressure hydrocephalus, Huntington's disease and Wernicke-Korsakoff syndrome, and any combinations thereof.

In some embodiments, the disease is a disease associated with deficient cognitive performance. In some embodiments, the disease is anxiety, depression, memory loss, or PTSD.

In some embodiments, the present disclosure provides methods for maintaining or enhancing muscle performance or muscle growth in a subject. Muscle performance may include, without limitation, the capacity of a muscle or a group of muscles to generate forces to produce, maintain, sustain, and modify postures and movements that are prerequisite to functional activities. In some embodiments, the method maintains or enhances muscle strength, muscle endurance, speed, muscle power, maximum muscle length, or oxygen level in muscle. In some embodiments, the method maintains muscle performance after or during physical labor or exercise. In some embodiments, the method reduces muscle fatigue, muscle sourness, or muscle tension after physical labor or exercise. In some embodiments, the method reduces the muscle reaction time after stimulation. In some embodiments, the method increases muscle mass or mitochondrial mass in muscle. In some embodiments, the method maintains or increases the number of myofibrils and rate of protein synthesis. In some embodiments, the method reduces inflammatory response or oxidative stress in muscle. In some embodiments, the subject has a deficiency in muscle performance or muscle growth. In some embodiments, the subject does not have a deficiency in muscle performance or muscle growth.

The present disclosure is also directed to a method of extending lifespan. In certain embodiments, the method maintains or enhances the healthspan in a subject with respect to body-mass index, physical performance, cardiac health, sexual performance, mental health, diet, or substance uses. Physical performance may include athletic performance and general fitness. Physical performance may refer to the capacity to complete daily round of activities with enough energy left for recreation and relaxation. Cardiac health may include health of the heart and blood vessels. Mental health may include emotional, psychological, and social well-being.

In another aspect, the methods provided herein may induce an increase of the number of mitochondria present in the subject (e.g., C. elegans or yeast). In some embodiments, the increase is greater than about 10%, or greater than about 20%, or greater than about 30%, or greater than about 40%, or greater than about 50%, or greater than about 60%, or greater than about 70%, or greater than about 80%, or greater than about 90%, or greater than about 100%, or greater than about 250%, or greater than about 500%, or greater than about 750%, or greater than about 1000%, or greater than 2000%. In some embodiments, the increase is about 10%-25%, about 25%-50%, about 50%-100%, about 100%-250%, about 250%-500%, about 500%-750%, about 750%-1000%, about 1000%-1500%, about 1500%-2000%, about 10%-100%, about 100%-500%, about 500%-2000%, or about 10%-2000%.

In another aspect, the methods provided herein may induce growth of the subject (e.g., C. elegans, yeast, or mammal). In some embodiments, the growth is in size of the subject. In some embodiments, the growth is in weight of the subject. In some embodiments, the growth is greater than about 10%, or greater than about 20%, or greater than about 30%, or greater than about 40%, or greater than about 50%, or greater than about 60%, or greater than about 70%, or greater than about 80%, or greater than about 90%, or greater than about 100%, or greater than about 250%, or greater than about 500%, or greater than about 750%, or greater than about 1000%, or greater than 2000%. In some embodiments, the growth is about 10%-25%, about 25%-50%, about 50%-100%, about 100%-250%, about 250%-500%, about 500%-750%, about 750%-1000%, about 1000%-1500%, about 1500%-2000%, about 10%-100%, about 100%-500%, about 500%-2000%, or about 10%-2000%.

In another aspect, the methods provided herein may induce an increase of the NAD+ levels in the subject (e.g., C. elegans, yeast, or mammal). In some embodiments, the increase is greater than about 10%, or greater than about 20%, or greater than about 30%, or greater than about 40%, or greater than about 50%, or greater than about 60%, or greater than about 70%, or greater than about 80%, or greater than about 90%, or greater than about 100%, or greater than about 250%, or greater than about 500%, or greater than about 750%, or greater than about 1000%, or greater than 2000%. In some embodiments, the increase is about 10%-25%, about 25%-50%, about 50%-100%, about 100%-250%, about 250%-500%, about 500%-750%, about 750%-1000%, about 1000%-1500%, about 1500%-2000%, about 10%-100%, about 100%-500%, about 500%-2000%, or about 10%-2000%.

In another aspect, the methods provided herein may induce a decrease of the reactive oxygen species (ROS) or a decrease of oxidative stress in the subject (e.g., C. elegans, yeast, or mammal). Examples of ROS include, without limitation, peroxides, superoxide, singlet oxygen, hydroxyl radicals, and alpha-oxygen. In some embodiments, the ROS is from exogenous sources. In some embodiments, the ROS is from endogenous sources. In some embodiments, the decrease of ROS is greater than about 10%, or greater than about 20%, or greater than about 30%, or greater than about 40%, or greater than about 50%, or greater than about 60%, or greater than about 70%, or greater than about 80%, or greater than about 90%, or greater than about 100%, or greater than about 250%, or greater than about 500%, or greater than about 750%, or greater than about 1000%, or greater than 2000%. In some embodiments, the decrease of ROS is about 10%-25%, about 25%-50%, about 50%-100%, about 100%-250%, about 250%-500%, about 500%-750%, about 750%-1000%, about 1000%-1500%, about 1500%-2000%, about 10%-100%, about 100%-500%, about 500%-2000%, or about 10%-2000%.

In some embodiments, oxidative stress can be measured by levels of any oxidative stress biomarkers known in the art. Examples of oxidative stress biomarkers include, without limitation, lipid peroxidation (e.g., thiobarbituric acid-reactive substances or oxidized low-density lipoprotein (LDL)), protein oxidation (e.g., protein carbonyls or protein nitration), DNA oxidation (e.g., 8-oxo-7,8-dihydro-2′-deoxyguansine), superoxide dismutase, and glutathione system (e.g., glutathione peroxidase or reduced glutathione). In some embodiments, the level of one or more oxidative stress biomarkers is decreased by greater than about 10%, or greater than about 20%, or greater than about 30%, or greater than about 40%, or greater than about 50%, or greater than about 60%, or greater than about 70%, or greater than about 80%, or greater than about 90%, or greater than about 100%, or greater than about 250%, or greater than about 500%, or greater than about 750%, or greater than about 1000%, or greater than 2000%. In some embodiments, the level of one or more oxidative stress biomarkers is decreased by about 10%-25%, about 25%-50%, about 50%-100%, about 100%-250%, about 250%-500%, about 500%-750%, about 750%-1000%, about 1000%-1500%, about 1500%-2000%, about 10%-100%, about 100%-500%, about 500%-2000%, or about 10%-2000%.

In another aspect, the methods provided herein may induce an increase of muscle mass in the subject (e.g., mammal). In some embodiments, increase is greater than about 10%, or greater than about 20%, or greater than about 30%, or greater than about 40%, or greater than about 50%, or greater than about 60%, or greater than about 70%, or greater than about 80%, or greater than about 90%, or greater than about 100%, or greater than about 250%, or greater than about 500%, or greater than about 750%, or greater than about 1000%, or greater than 2000%. In some embodiments, the increase is about 10%-25%, about 25%-50%, about 50%-100%, about 100%-250%, about 250%-500%, about 500%-750%, about 750%-1000%, about 1000%-1500%, about 1500%-2000%, about 10%-100%, about 100%-500%, about 500%-2000%, or about 10%-2000%. In some embodiments, the subject is human.

### EXAMPLES

The following examples are offered to illustrate but not to limit the invention. One of ordinary skill in the art will recognize that the following procedures may be modified using methods known to one of ordinary skill in the art.

### Example 1: Assessment of Safety, Pharmacokinetics, and Pharmacodynamics of NAM, NA, NMN, and NR

Primary Endpoints:
1. Safety profile of NAM, NA, NMN, and NR.
2. Pharmacokinetics and pharmacodynamics.

Study Design
In this study, safety, pharmacokinetics, and pharmacodynamics of NAM, NA, NMN, and NR were studied. Before receiving the first oral dose of NAM, NA, NMN, and NR formulated as a dietary supplement, but after screening and informed consent, eligible subjects underwent blood and urine samplings on Day 1 to establish baseline levels of NAD+ and other compounds. Subsequently, each subject received one of the following dosages:
1. daily doses of 1000 mg NR for 7 days:
2. daily doses of NR 300 mg for 14 days and then added NAM 1000 mg for 7 days;
3. daily doses of 300 mg NR for 7 days;
4. daily doses of 1000 mg NAD+ for 7 days;
5. daily doses of 1000 mg NAM for 14 days:
6. daily doses of 300 mg NA for 14 days:
7. daily doses of 1000 mg NMN for 7 days:
8. daily doses of NAM, NA, NMN, and NR, one at a time, including 300 mg NA and 300 mg NR for 11 days followed by 300 mg NA, 300 mg NR, and 1000 mg NAM for 11 days;
9. daily doses of 300 mg NR and 1000 mg NAM for 9 days;
10. daily doses of NAM, NA, and NR, one at a time, including 300 mg NA and 300 mg NR for 11 days followed by 300 mg NA, 300 mg NR, and 1000 mg NAM for 11 days.
Each subject underwent serial blood and urine sampling during the daily dosing of NAM, NA, NMN, and NR, or a mixture of the foregoing.

Inclusion/Exclusion Criteria

Inclusion Criteria:
1. Healthy male or female, age>18 years.
2. Signed, informed consent.
3. Female of childbearing potential must have negative urine pregnancy test result and must agree to use adequate contraception (hormonal or barrier method of birth control or abstinence) during the study and one month thereafter.
4. Healthy as determined by laboratory results and medical history.
5. Agrees to maintain current level of physical activity throughout the study.
6. Agrees to avoid study compounds for 30 days prior to enrollment and during the study 7.
7. Agrees to avoid nutritional yeast, whey proteins, energy drinks, pomegranate, pomegranate juice, grapefruit, grapefruit juice and alcohol 7 days prior to enrollment and during study
8. Willingness and ability to comply with scheduled visits, cell phone calls, treatment plans, laboratory tests, and completion of other study procedures as specified in the protocol.

Exclusion Criteria:
1. Women who were pregnant, breastfeeding, or planning to became pregnant during the course of the trial.
2. Subjects who were smokers
3. Unstable medical conditions as determined by the Investigator
4. Immunocompromised individuals such as subjects that had undergone organ transplantation or subjects diagnosed with human immunodeficiency virus (HIV)
5. Clinically significant abnormal lab results at screening (e.g., AST, ALT or ALP>2×ULN, and/or bilirubin>1×ULN)
6. Subjects who had planned surgery during the course of the trial
7. History of or current diagnosis of any cancer (except for successfully treated basal cell carcinoma) diagnosed less than 5 years prior to screening. Subjects with cancer in full remission more than 5 years after diagnosis were acceptable
8. History of blood/bleeding disorders
9. Blood donation in the past 2 months
10. Alcohol abuse (>2 standard alcoholic drinks per day) or drug abuse within the past 6 months
11. Participation in a clinical research trial within 30 days prior to randomization
12. Allergy or sensitivity to study supplement ingredients provided during the study
13. Individuals who were cognitively impaired and/or who were unable to give informed consent.
14. Any other condition which in the Investigator's opinion may adversely affect the subject's ability to complete the study or its measures or which may pose significant risk to the subject

Clinical Data and Sample Collections
1. Height, weight, temperature, BP, heart rate, demographics
2. Physical examination
3. Medical history, concomitant dietary supplements (if any) and concomitant medications (if any)
4. Plasma/blood samples for routine laboratory tests—CBC with platelet count and WBC differential, electrolytes, glucose, calcium magnesium, liver function tests (Alanine transaminase (ALT), aspartate aminotransferase (AST), alkaline phosphatase (ALP), bilirubin, creatinine, Albumin), lactate dehydrogenase.
5. Adverse events

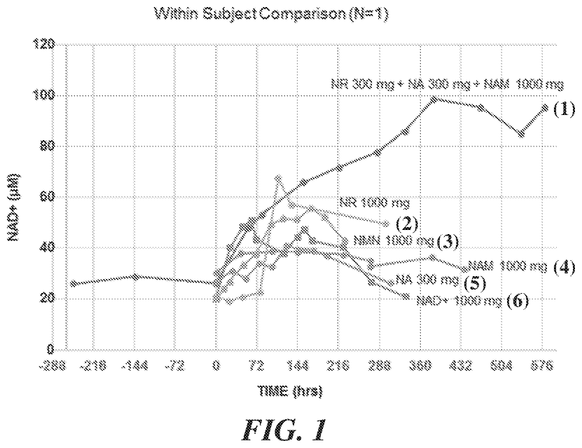

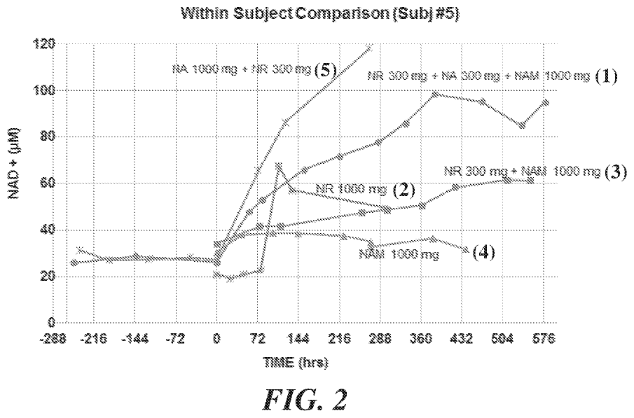

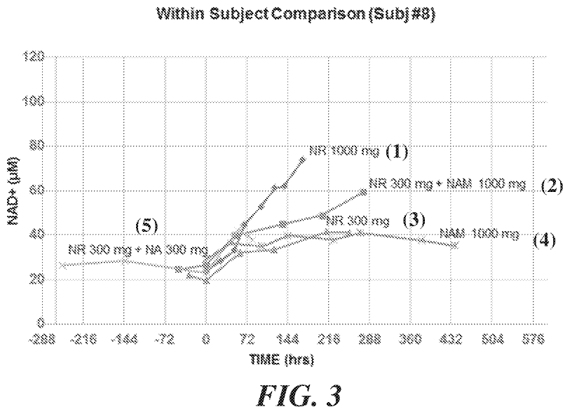

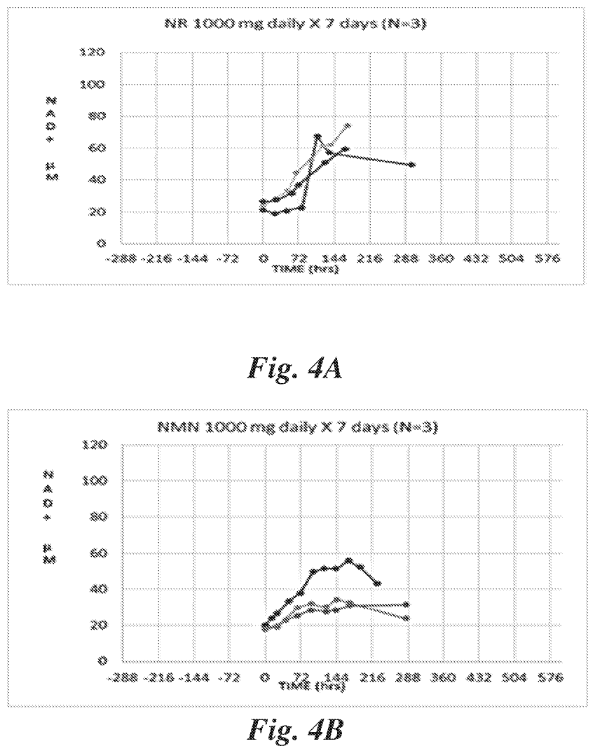

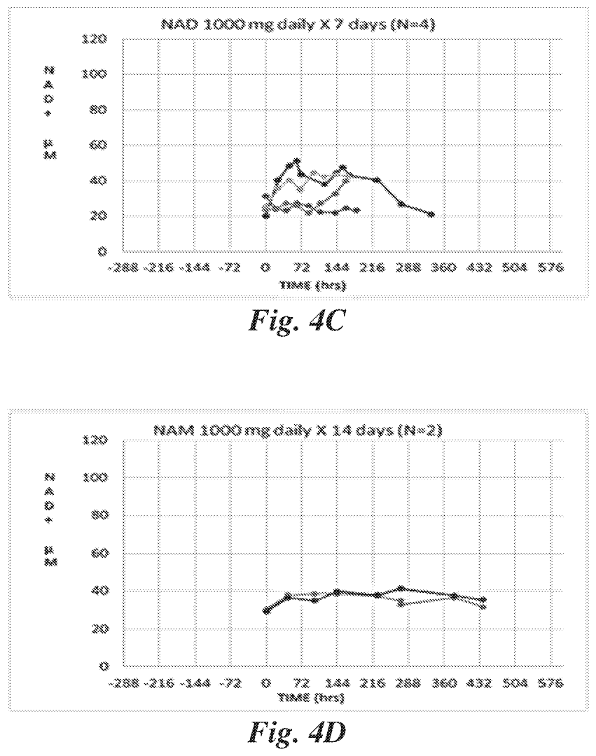

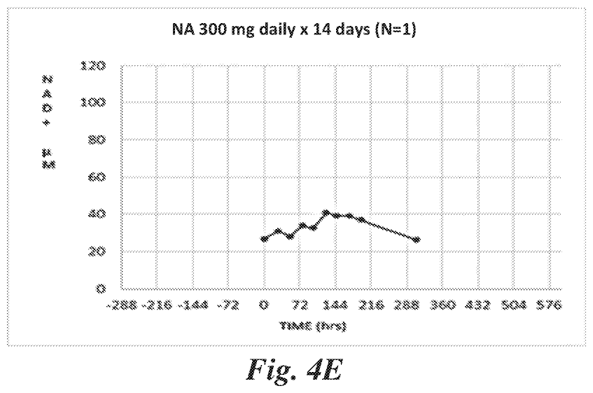

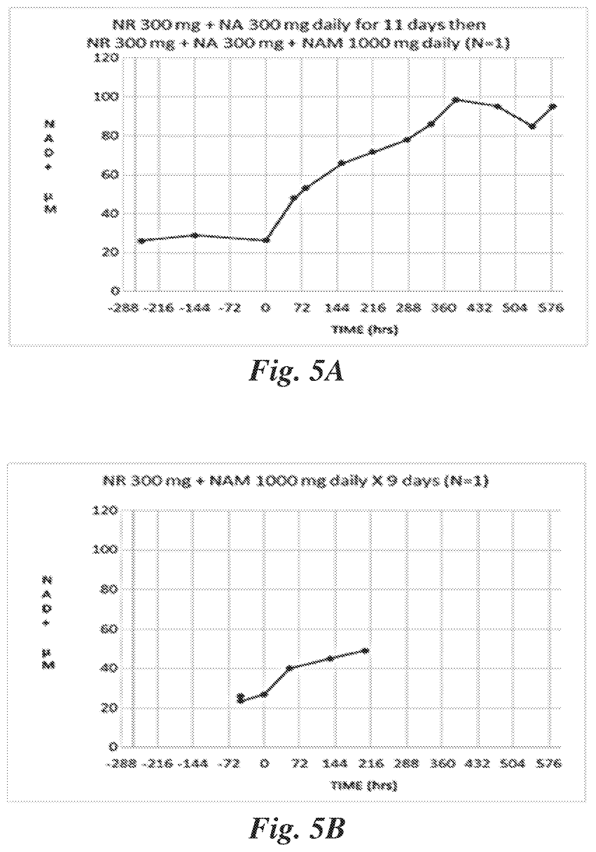

Results
1. Clinical Evaluations:
   a. Blood draws were obtained at the start and end of the study to determine changes in the health of the patient over the course of the study. Apart from measuring NAD+ levels in the blood, samples taken on baseline visit and final visit were analyzed for routine laboratory tests—CBC with platelet count and WBC differential, electrolytes, glucose, calcium, magnesium, liver function tests (Alanine transaminase (ALT), aspartate aminotransferase (AST), alkaline phosphatase (ALP), bilirubin, creatinine, Albumin), lactate dehydrogenase.
   b. For the serial blood sampling, finger prick method were used to minimize discomfort to the patient and reduce the volume of blood required. Blood draws will be performed by qualified nurses, physicians or phlebotomists according to the standard phlebotomy techniques.
2. FIGS. 1, 2, 3, 4A-4E, and 5A-5B show the data collected in this study and have demonstrated modulations of NAD+ levels by these NAD+-related compounds.

### Example 2. Assessment of Safety, Pharmacokinetics, and Pharmacodynamics of NAD+-Related Compounds

Primary Endpoints:
1. Safety profile of the combination of two or more active NAD+-related compounds
2. Pharmacokinetics and pharmacodynamics.

Study Design

In this study, safety, pharmacokinetics and pharmacodynamics of NAD+-related compounds are investigated. Before receiving the first oral dose of NAD+-related compounds formulated as a dietary supplement or a pharmaceutical composition, but after screening and informed consent, eligible subjects undergo blood and urine samplings on Day 1 to establish baseline levels of NAD+, muscle mass and oxidative stress biomarkers. Subsequently, each subject receives one of the following dosages:
1. daily doses of 1000 mg NR for 7 days:
2. daily doses of NR 300 mg for 14 days and then added NAM 1000 mg for 7 days:
3. daily doses of 300 mg NR for 7 days:
4. daily doses of 1000 mg NAD+ for 7 days;
5. daily doses of 1000 mg NAM for 14 days;
6. daily doses of 300 mg NA for 14 days;
7. daily doses of 100 mg NMN for 7 days;
8. daily doses of 1000 mg NADP+ for 7 days:
9. daily doses of NAM, NA, NMN, and NR, one at a time, including 300 mg NA and 300 mg NR for 11 days followed by 300 mg NA, 300 mg NR, and 1000 mg NAM for 11 days;
10. daily doses of 300 mg NR and 1000 mg NAM for 9 days.
11. daily doses of NAM, NAD+, NMN, and NR, one at a time, including 300 mg NA and 300 mg NR for 11 days followed by 300 mg NAD+, 300 mg NR, and 1000 mg NAM for 11 days:
12. daily doses of NAM, NAR, NMN, and NR, one at a time, including 300 mg NA and 300 mg NR for 11 days followed by 300 mg NAR, 300 mg NR, and 1000 mg NAM for 11 days;
13. daily doses of NAM, NA, NMN, inositol hexanicotinate, and NR, one at a time, including 300 mg NA and 300 mg NR for 11 days followed by 300 mg NA, 300 mg NR, 300 mg inositol hexanicotinate, and 1000 mg NAM for 11 days:
14. daily doses of NAM, NAD+, NMN, inositol hexanicotinate, and NR, one at a time, including 300 mg NA and 300 mg NR for 11 days followed by 300 mg NAD+, 300 mg NR, 300 mg inositol hexanicotinate, and 1000 mg NAM for 11 days;
15. daily doses of NAM, NAR, NMN, inositol hexanicotinate, and NR, one at a time, including 300 mg NA and 300 mg NR for 11 days followed by 300 mg NAR, 300 mg NR, 300 mg inositol hexanicotinate, and 1000 mg NAM for 11 days;

Each subject underwent serial blood and urine sampling during the daily dosing of NAM, NA, NMN, NAD+, NAR, inositol hexanicotinate, NR, or a mixture of the foregoing.

Each subject undergoes serial blood and urine sampling during the daily dosing of NAD+-related compounds, or a mixture of the foregoing. If applicable, each subject goes through a two-week washing period in which no NAD+-related compounds are given before the subject is eligible for testing a different combination.

Inclusion/Exclusion Criteria are the same as Example 1.

Clinical Data and Sample Collections
Height, weight, temperature, BP, heart rate, demographics
NAD+ levels, muscle mass, and oxidative stress biomarkers including lipid peroxidation levels (e.g., thiobarbituric acid-reactive substances or oxidized low-density lipoprotein (LDL)), protein oxidation levels (e.g., protein carbonyls or protein nitration), DNA oxidation levels (e.g., 8-oxo-7,8-dihydro-2′-deoxyguansine), superoxide dismutase levels, and glutathione system (e.g., glutathione peroxidase or reduced glutathione).
Adverse events

### Example 3. Effects of NAD+-Related Compounds in C. elegans and Yeast

The effects of the NAD+-related compounds combinations on model organism c. elegans and yeast are studied. C. elegans strains are cultured at 20° C. on nematode growth media agar plates and are exposed to different compound combinations during the full life from eggs until death. After the exposure, C. elegans are measured for the growth in size and mass, and their lifespan is recorded. Mitochondria are isolated and measured to estimate the increase in number using the method described, for example, in Ryu et al. (2016) Nat. Med. 22, 879.

Yeast strains, such as Saccharomyces cerevisiae, are grown in glucose medium and exposed to different compound combinations. Replicative life span is determined using the method described in Sinclair et al. Cell (1997) 91, 1033.

Particular agents and combinations thereof administered to the organisms are summarized below:
1. daily doses of 1000 mg NR for 7 days;
2. daily doses of NR 300 mg for 14 days and then added NAM 1000 mg for 7 days;
3. daily doses of 300 mg NR for 7 days;
4. daily doses of 1000 mg NAD+ for 7 days;
5. daily doses of 1000 mg NAM for 14 days;
6. daily doses of 300 mg NA for 14 days:
7. daily doses of 100 mg NMN for 7 days;
8. daily doses of 1000 mg NADP+ for 7 days;
9. daily doses of NAM, NA, NMN, and NR, one at a time, including 300 mg NA and 300 mg NR for 11 days followed by 300 mg NA, 300 mg NR, and 1000 mg NAM for 11 days;
10. daily doses of 300 mg NR and 1000 mg NAM for 9 days.
11. daily doses of NAM, NAD+, NMN, and NR, one at a time, including 300 mg NA and 300 mg NR for 11 days followed by 300 mg NAD+, 300 mg NR, and 1000 mg NAM for 11 days;
12. daily doses of NAM, NAR, NMN, and NR, one at a time, including 300 mg NA and 300 mg NR for 11 days followed by 300 mg NAR, 300 mg NR, and 1000 mg NAM for 11 days;
13. daily doses of NAM, NA, NMN, inositol hexanicotinate, and NR, one at a time, including 300 mg NA and 300 mg NR for 11 days followed by 300 mg NA, 300 mg NR, 300 mg inositol hexanicotinate, and 1000 mg NAM for 11 days;
14. daily doses of NAM, NAD+, NMN, inositol hexanicotinate, and NR, one at a time, including 300 mg NA and 300 mg NR for 11 days followed by 300 mg NAD+, 300 mg NR, 300 mg inositol hexanicotinate, and 1000 mg NAM for 11 days;
15. daily doses of NAM, NAR, NMN, inositol hexanicotinate, and NR, one at a time, including 300 mg NA and 300 mg NR for 11 days followed by 300 mg NAR, 300 mg NR, 300 mg inositol hexanicotinate, and 1000 mg NAM for 11 days.

What is claimed is:

1. A composition comprising two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

2. The composition of claim 1, wherein at least one of the two or more active agents is at least about 80% pure, wherein the % purity excludes the weight of any other active agents and any added vehicle, diluent, excipient, or carrier.

3. The composition of claim 1 or claim 2, wherein the composition is sterilized.

4. The composition of any one of claims 1-3, wherein the composition comprises nicotinamide riboside or a salt thereof and nicotinamide mononucleotide or a salt thereof.

5. The composition of claim 4, wherein the composition further comprises nicotinic acid or a salt thereof.

6. The composition of any one of claims 1-5, further comprising salicylic acid or a salt thereof, or acetylsalicylic acid or a salt thereof.

7. The composition of any one of claims 1-6, wherein the composition is formulated for oral, topical, intramuscular, intravenous, intrabuccal, or sublingual administration to a subject.

8. The composition of any one of claims 1-7, wherein the composition is in the form of a tablet, a capsule, an extended-release tablet, a liquid, a powder, granules, a dragee, or a lozenge.

9. The composition of any one of claims 1-6, wherein the composition is in the form of a food product or dietary supplement.

10. The composition of any one of claims 1-8, further comprising a pharmaceutically acceptable carrier, excipient, binder, or diluent.

11. The composition of claim 10, further comprising a wax matrix.

12. The composition of any one of claims 1-11, wherein the two or more active agents are present in the amount of about 300-3000 mg.

13. A pharmaceutical composition comprising a therapeutically effective amount of the composition of any one of claims 1-12, or a pharmaceutically acceptable salt thereof, and a pharmaceutically acceptable carrier, excipient, binder, or diluent.

14. A dietary supplement or food product comprising the composition of any one of claims 1-12 or the pharmaceutical composition of claim 13.

15. A kit comprising two or more components, wherein each component comprises one or more active agents independently selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

16. The kit of claim 15, wherein at least one active agent is at least about 80% pure, wherein the % purity excludes the weight of any other active agents and any added vehicle, diluent, excipient, or carrier.

17. The kit of claim 15 or 16, wherein at least one of the two or more components is sterilized.

18. The kit of claim 15, wherein each of the active agents is at least about 80% pure, wherein the % purity excludes the weight of any other active agents and any added vehicle, diluent, excipient, or carrier.

19. The kit of claim 17 or 18, wherein the two or more components are each sterilized.

20. The kit of any one of claims 15-19, wherein the active agents comprise nicotinamide riboside or a salt thereof and nicotinamide mononucleotide or a salt thereof.

21. The kit of claim 20, wherein the active agents further comprise nicotinic acid or a salt thereof.

22. The kit of any one of claims 15-21, further comprising salicylic acid or a salt thereof, or acetylsalicylic acid or a salt thereof.

23. The kit of any one of claims 15-22, wherein the kit comprises a first active agent and a second active agent, wherein the ratio of the first active agent and the second active agent is about 1:1 to 25:1 (w/w).

24. The kit of any one of claims 15-22, wherein the kit comprises a first active agent, a second active agent, and a third active agent, wherein the ratio of the first active agent and the second active agent is about 1:1 to 25:1 (w/w) and the ratio of the first active agent and the third active agent is about 1:1 to 25:1 (w/w).

25. The kit of any one of claims 15-24, wherein at least one of the two or more components further comprises a pharmaceutically acceptable carrier, excipient, binder, or diluent.

26. The kit of claim 25, wherein at least one of the two or more components further comprises a wax matrix.

27. The kit of any one of claims 15-26, wherein one or more components are formulated for oral, topical, intramuscular, intravenous, intrabuccal, or sublingual administration to a subject.

28. The kit of any one of claims 15-27, wherein one or more components are in the form of a tablet, a capsule, an extended-release tablet, a liquid, a powder, granules, a dragee, or a lozenge.

29. The kit of any one of claims 15-28, wherein one or more components are in the form of a food product or dietary supplement.

30. The kit of any one of claims 15-29, wherein the two or more components are formulated for simultaneous administration to a subject.

31. The kit of any one of claims 15-29, wherein the two or more components are formulated for sequential administration to a subject.

32. The kit of any one of claims 15-31, wherein the two or more components are present in the amount of about 300-3000 mg.

33. A method of modulating blood NAD+ levels comprising administering to a subject an effective amount of the composition of any of claims 1-12, the pharmaceutical composition of claim 13, or the dietary supplement or food product of claim 14.

34. A method of extending lifespan of a subject comprising administering to the subject an effective amount of the composition of any of claims 1-12, the pharmaceutical composition of claim 13, or the dietary supplement or functional food of claim 14.

35. A method of improving healthspan of a subject comprising administering to the subject an effective amount of the composition of any of claims 1-12, the pharmaceutical composition of claim 13, or the dietary supplement or functional food of claim 14.

36. A method of enhancing or maintaining muscle growth or performance comprising administering to a subject an effective amount of the composition of any of claims 1-12, the pharmaceutical composition of claim 13, or the dietary supplement or functional food of claim 14.

37. A method of treating or preventing a mitochondrial disease or condition comprising administering to a subject in need thereof a therapeutically effective amount of the composition of any of claims 1-12, the pharmaceutical composition of claim 13, or the dietary supplement or functional food of claim 14.

38. A method of modulating blood NAD+ levels in a subject comprising co-administering to the subject a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

39. A method of extending lifespan of a subject comprising co-administering to a subject a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

40. A method of improving healthspan of a subject comprising co-administering to a subject a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

41. A method of enhancing or maintaining muscle growth or performance comprising co-administering to a subject in need thereof a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

42. A method of treating or preventing a mitochondrial disease comprising co-administering to a subject in need thereof a therapeutically effective amount of two or more active agents selected from the group consisting of NAD+, NADH, NADP+, NADPH, nicotinic acid adenine dinucleotide, nicotinic acid mononucleotide, nicotinamide, nicotinic acid, nicotinamide mononucleotide, nicotinic acid riboside, nicotinamide riboside, inositol hexanicotinate, a salt of any of the foregoing, and a mixture of any of the foregoing.

43. The method of any one of claims 38-42, wherein at least one of the two or more active agents is at least about 80% pure, wherein the % purity excludes the weight of any other active agents and any added vehicle, diluent, excipient, or carrier.

44. The method of any one of claims 38-43, wherein at least one of the two or more active agents is sterilized.

45. The method of any one of claims 38-44, wherein the method comprises co-administering to the subject a therapeutically effective amount of nicotinamide riboside or a salt thereof and nicotinamide mononucleotide or a salt thereof.

46. The method of claim 45, wherein the method comprises administering nicotinic acid or a salt thereof.

47. The method of any one of claims 38-46, further comprising administering salicylic acid or a salt thereof, or acetylsalicylic acid or a salt thereof.

48. The method of claim 47, wherein the salicylic acid or a salt thereof, or acetylsalicylic acid or a salt thereof is administered prior to the co-administration of the two or more active agents.

49. The method of any one of claims 38-48, wherein the method comprises co-administering of a first active agent and a second active agent, wherein the ratio of the first active agent and the second active agent is about 1:1 to 25:1 (w/w).

50. The method of any one of claims 38-49, wherein the method comprises co-administering of a first active agent, a second active agent, and a third active agent, wherein the ratio of the first active agent and the second active agent is about 1:1 to 25:1 (w/w) and the ratio of the first active agent and the third active agent is about 1:1 to 25:1 (w/w).

51. The method of any one of claims 38-50, wherein at least one of the two or more active agents is formulated for oral, topical, intramuscular, intravenous, intrabuccal, or sublingual administration to a subject.

52. The method of any one of claims 38-51, wherein at least one of the two or more active agents is in the form of a tablet, a capsule, an extended-release tablet, a liquid, a powder, granules, a dragee, or a lozenge.

53. The method of any one of claims 38-52, wherein at least one of the two or more active agents is in the form of a food product or dietary supplement.

54. The method of any one of claims 38-53, wherein the two or more active agents are formulated for simultaneous administration to a subject.

55. The method of any one of claims 38-53, wherein the two or more active agents are formulated for sequential administration to a subject.

56. The method of any one of claims 38-55, wherein the two or more active agents are present in the amount of about 300-3000 mg.

57. The method of any one of claims 38-56, wherein the NAD+ level in the subject increases by greater than about 50%.

58. The method of any one of claims 38-56, wherein the level of reactive oxygen species in the subject is reduced by greater than about 50%.
